(19) United States
(12) Patent Application Publication
(10) Pub. No.: US 2008/0208231 A1
(43) Pub. Date: Aug. 28, 2008

(54) HANDPIECE AND HORN FOR ULTRASONIC SURGICAL INSTRUMENT

(75) Inventors: Hidefumi OTA, Tokyo (JP); Tomohiro Shibata, Tokyo (JP); Yuichiro Sato, Tokyo (JP)

Correspondence Address: YOUNG & THOMPSON, 209 Madison Street, Suite 500, ALEXANDRIA, VA, 22314, US

(73) Assignee: MIWATEC CO., LTD, TOKYO (JP)

(21) Appl. No.: 11/938,936

(22) Filed: Nov. 13, 2007

(30) Foreign Application Priority Data
Nov. 13, 2006 (JP) 2006-306694

(51) Int. Cl. A61B 17/32

(52) U.S. Cl. 606/169

(57) ABSTRACT

The present invention aims to provide a handpiece and a horn for an ultrasonic surgical instrument in which better visual recognition of a tip portion of the horn can be given. The handpiece for an ultrasonic surgical instrument includes: a horn of which tip portion vibrates at an ultrasonic wave velocity; and an external cylinder for covering the horn except the tip portion. An external diameter of the tip portion of the horn is larger than an external diameter of a part covered by the external cylinder.

This application is based upon and claims the benefit of priority from Japanese patent application No. 2006-308529, filed on Nov. 15, 2006, the disclosure of which is incorporated herein in its entirety by reference.

### BACKGROUND OF THE INVENTION

1. Field of the Invention

The present invention relates to a handpiece and a horn of an ultrasonic surgical instrument using ultrasonic vibration.

2. Description of the Related Art

In medical treatment, conventionally, as one of various surgical instruments in the surgical field, many ultrasonic handpieces have been used. FIG. 1 shows such ultrasonic handpiece A.

In FIG. 1, external cylinder 1 contains an ultrasonic vibration structure including a vibrator of, for example, a magneto-striction type and an electrostriction type, and which outputs ultrasound having a predetermined frequency. Horn 2 fits in an opening in one end portion of the external cylinder by insertion, and cuts hard tissues such as bone with the tip portion thereof by using vibration transmitted from the ultrasonic vibration structure. Joint 1a is attached to a tube for sucking in, for example, irrigation water and a chip. Joint 1b is attached to a tube for injecting the irrigation water for dissipate heat in the tip portion generated by vibration and for cooling the frictional heat generated at the time of cutting the bone. Cable 1c supplies high frequency electric energy to the ultrasonic vibration structure.

Horn 2 vibrates in the axial direction thereof with a predetermined frequency due to vibrations transmitted from the ultrasonic vibration structure, and cuts the required place with a tip that abuts against hard tissues such as the bone.

FIG. 2 is a cross-section view illustrating an internal structure of external cylinder 1 and horn 2.

External cylinder 1 is configured to cover horn 2 including a vibration generator.

The vibration generator includes flange 8, piezoelectric elements 9 and 10, electrodes 11 and 12, front plate 13, and backing plate 14.

Piezoelectric elements 9 and 10 are positioned between front plate 13 and backing plate 14, and generate vibration in the horizontal direction shown in FIG. 2 correspondingly to the high frequency power supply applied to electrodes 11 and 12 through cable 1c shown in FIG. 8. Front plate 13 is integrally formed with flange 8. Also, a support member not shown is provided on backing plate 14 on the side opposite to piezoelectric element 10. Flange 8 and the support member described above hold each of piezoelectric elements 9 and 10, front plate 13, and backing plate 14 in external cylinder 1, and thereby the direction of vibration is controlled.

The components described above respectively include a screw in the joint portions, and are integrally coupled with each other by screwing them together.

The tip portion of horn 2 protruding from external cylinder 1 actually abuts against an affected area. The amount of vibration (movement) of the tip portion is large, so the heat quantity becomes extremely large.

Also, an ultrasonic surgical instrument fragments, emulsifies and sucks human tissues by using ultrasonic vibration to selectively ablate an affected area. Therefore, in the periphery of external cylinder 1, joint 1b for injecting irrigation water for cooling the tip of the tip portion and for facilitating suction of an ablated part is provided.

In horn 2, front plate 13, piezoelectric elements 9 and 10, backing plate 14, and external cylinder 1 configured as described above, intake port 15 are provided in a linked manner along the center line. The fragmented and emulsified tissues are sucked out through intake port 15 and joint 1a shown in FIG. 8 by a suction pump externally provided. Further, each of the components described above is formed in an approximately rotational symmetry around intake port 15 as the axis.

Also, vibration conversion structure 17 is provided in horn 2. Vibration conversion structure 17 is the same as the one that was applied by the applicant and disclosed in published Patent Document 1 (Japanese Patent Application Laid-Open No. 2005-152098).

FIG. 3 shows the details of vibration conversion structure 17. As shown in FIG. 3, vibration conversion structure 17 is configured using a plurality of grooves 17a formed to wind around the outer surface of horn 2.

The plurality of grooves 17a are respectively engraved in parallel with a predetermined distance, and have predetermined deflection angle α relative to central axis X-X of horn 2 on the outer surface, and this angle α is set to be in the range of 0°<α<90°.

Also, grooves 17a are rectangular in shape, and their width is set to 0.5 to 5 mm, their length is set to 3 to 30 mm, and their depth is set to be in a range not smaller than 0.5 mm.

In addition, the position at which grooves are set in vibration conversion structure 17 is not limited to the outer surface of horn 2, but the grooves may be provided on either an external surface of horn 2 between the tip of horn 2 and an electrostriction element of a sound wave oscillation structure, or an external surface of a member provided in the sound wave oscillation structure or placed between horn 2 and the sound wave oscillation structure.

FIG. 4 shows operation of horn 2 in the tip. While the tip of ultrasonic horn 2 moves around the central axis forward and backward at a high speed (torsional vibration) in the direction shown by arrow A, the tip further reciprocates at a high speed (longitudinal vibration) along the central axis in the direction shown by arrow B, due to combination of the longitudinal vibration and the torsional vibration generated by conversion of longitudinal vibration in vibration conversion structure 17.

An effect of vibration conversion by the grooves, now, may be considered as follows. Grooves 17a, as shown in FIG. 3, repeat deformation due to the longitudinal vibration, and it is thought that, at the time of deformation, a part of a component in the longitudinal direction is converted to a component in the torsional direction.

As configured in the above manner, high speed forward and backward rotation and reciprocation are combined in the tip of horn 2. Accordingly, shear efficiency of tissues is considerably improved, and further acuteness in cutting action, that is, so-called “sharpness” is also enhanced dramatically, and thereby crush etc. is not caused in the tissues of an ablated site, and cutting in a clean situation can be realized.

At the time of use, the tip of horn 2 is pressed against an affected area to fragment and emulsify tissues of the affected area. At this time, irrigation water injected through joint 1b cools horn 2 when it passes through the gap between external cylinder 1 and horn 2, and after being discharged from external cylinder 1, the irrigation water along with an ablated part is sucked into intake port 15 and discharged outside.

FIG. 5A is an outline view of the tip portion of horn 2 protruding from external cylinder 1 and abutting against an affected area in the conventional hand piece for an ultrasonic surgical instrument described above, and FIG. 5B is a cross-section view thereof. As shown, the external diameter of horn 2 is set to be constant and smaller than the inside diameter of external cylinder 1. A gap produced between the inside diameter of external cylinder 1 and the external diameter of horn 2 is set to be sufficiently large. It is because it is necessary to supply irrigation water to the tip of horn 2 through this gap, that horn 2 vibrates, and when external cylinder 1 and horn 2 come into contact with each other because of the narrow gap, frictional heat is generated to cause a risk of damage to external cylinder 1 or horn 2, so such circumstances are prevented from occurring.

From the reason described above, the external diameter of horn 2 protruding from external cylinder 1 has been set to be sufficiently smaller than the external diameter of external cylinder 1. As a result, at the time when the tip portion of horn 2 abutting against an affected area of the body is visually recognized, external cylinder 1 may form an obstacle depending on the surgical site may prevent good visual recognition of the tip portion of horn 2.

Because, in an operation, the tip portion of horn 2 is brought into contact with an affected area in the body, visual recognition is given in the longitudinal direction of the backside of handpiece. FIG. 6 illustrates an angle of visibility at the time of visual recognition of the tip of horn 2.

As shown in FIG. 6, to secure good field of view, it is necessary to make a smaller angle θ relative to the longitudinal direction of the handpiece, but when operating in a narrow place, a sufficient angle θ may not be secured depending on a surgical site.

The problem of visibility described above becomes severe especially when an ultrasonic surgical instrument is used to ablate a tumor. When a tumor is ablated, an operation is performed in a state in which only the flat surface of the end face of the tip portion abuts against an affected area. Accordingly, when the external diameter of horn 2 protruding from external cylinder 1 is smaller than the external diameter of external cylinder 1, it becomes difficult to directly confirm the situation of the affected area.

Also, as shown in the conventional example in FIG. 1, when irrigation water is used and when there is complex torsional and longitudinal vibration of horn 2, irrigation water 110 flowing out from a gap between external cylinder 1 and horn 2, as shown in FIG. 7, splashes off in the circumferential direction of horn 2, which may also block visual recognition

### SUMMARY OF THE INVENTION

The present invention was made in view of the problem that the conventional art has had as described above, and an object thereof is to provide a handpiece and a horn for an ultrasonic surgical instrument in which better visual recognition of a tip portion of the horn can be provided.

A handpiece for an ultrasonic surgical instrument of the present invention includes: a horn in which a flat surface is formed on the end face of a tip portion; and an external cylinder for covering the horn except the tip portion, in which the external diameter of the tip portion of the horn is larger than the external diameter of the part covered by the external cylinder.

In this case, the external diameter of the tip portion of the horn may be larger than the external diameter of the external cylinder.

Further, the horn may be configured so that the tip portion and the part covered by the external cylinder are formed to have a smooth, curved surface.

Also, the horn may be configured so that a step is formed in a joint portion between the tip portion and the part covered by the external cylinder.

Further, the horn may be configured so that the joint portion between the tip portion and the part covered by the external cylinder has a tapered shape in which the tip portion is bored in a conical shape.

Further, a groove may be formed on the end face of the tip portion.

Also, a nick may be formed on the flat surface.

A horn of the present invention includes a flat surface formed on an end face of a tip portion and constitutes the handpiece for an ultrasonic surgical instrument along with an external cylinder for covering the horn except the tip portion, in which an external diameter of the tip portion is larger than the external diameter of the part covered by the external cylinder.

In this case, the tip portion and the part covered by the external cylinder may be formed to have a smooth, curved surface.

Also, a step may be formed in a joint portion between the tip portion and the part covered by the external cylinder.

Further, the joint portion between the tip portion and the part covered by the external cylinder may have a tapered shape in which the tip portion is bored in a conical shape.

Also, a vibration conversion structure for converting ultrasonic, longitudinal vibration into ultrasonic, torsional vibration may be provided.

Further, a groove may be formed on the end face of the tip portion.

Also, a nick may be formed on the flat surface.

The present invention has the following advantages because of the configuration as described above.

Visibility of the tip portion of the horn can be improved because the external diameter of the tip portion of the horn is larger than the external diameter of the part covered by the external cylinder. This advantage is further improved by making the external diameter of the tip portion of the horn larger than the external diameter of the external cylinder.

Irrigation water flows out from the joint potion between the tip portion of the horn and the part covered by the external cylinder. The joint portion has a smooth, curved surface, a step, or an inverse, tapered shape, and therefore the direction in which irrigation water is reflected on the joint portion turns out to be the direction in which splashing of the irrigation water in the circumferential direction is suppressed, and thereby the amount of splash can be reduced, further improving visibility.

### BRIEF DESCRIPTION OF THE DRAWINGS

FIG. 1 shows an ultrasonic handpiece;

FIG. 2 is a cross-section view illustrating an internal structure of external cylinder 1 and horn 2 in FIG. 1;

FIG. 3 shows the details of vibration conversion structure 17 in FIG. 2;

FIG. 4 shows operation of horn 2 in a tip;

FIGS. 5A and 5B, respectively, are an outline view and a cross-section view of the tip portion of horn 2 protruding from external cylinder 1 and abutting against an affected area;

FIG. 6 illustrates the angle of visibility when a conventional example shown in FIG. 5 is used for an operation;

FIG. 7 shows the direction in which irrigation water splashes off in the conventional example shown in FIG. 5;

FIGS. 8A and 8B, respectively, show the main configuration of one embodiment of a handpiece for an ultrasonic surgical instrument according to the present invention;

FIG. 9 illustrates the angle of visibility when the embodiment shown in FIGS. 8A and 8B is used for an operation;

FIG. 10 illustrates a state of splashing of irrigation water in the embodiment shown in FIGS. 8A and 8B, and is a cross-section view of an enlarged part surrounded by the dotted line in FIG. 8B;

FIGS. 11A and 11B, respectively, are cross-section views illustrating a configuration of other embodiments of the present invention, and similarly to FIG. 10, illustrate a state of splashing of irrigation water in each embodiment, and are cross-section views of an enlarged part surrounded by the dotted line in FIG. 8B; and

FIG. 12 is a perspective view illustrating a configuration of another embodiment of the present invention.

### EXEMPLARY EMBODIMENT

Then, an embodiment of the present invention will be described with reference to the accompanying drawings.

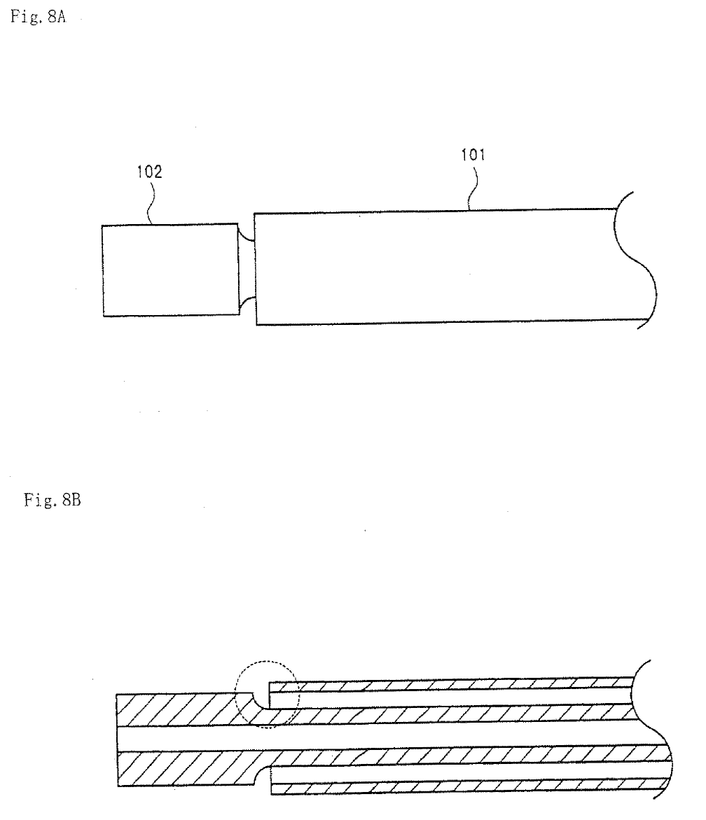

FIGS. 8A and 8B show the main configuration of one embodiment of a handpiece for an ultrasonic surgical instrument according to the present invention.

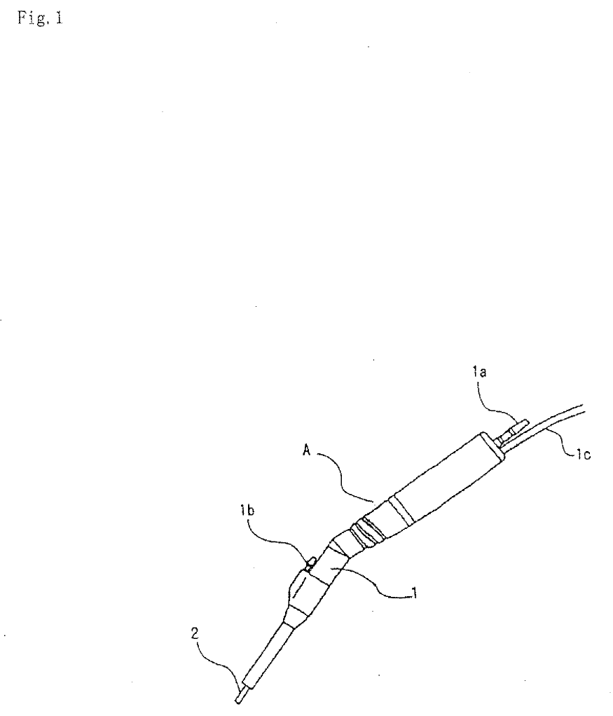

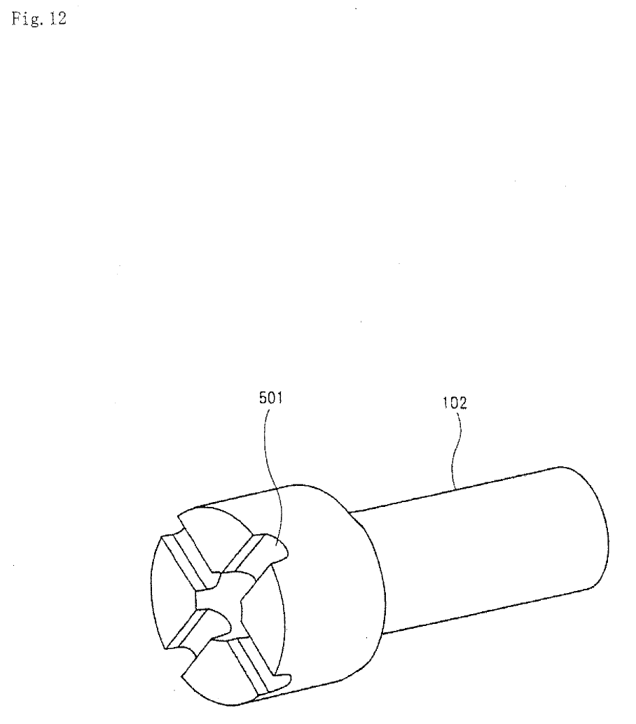

In the present embodiment, external cylinder 1 and horn 2 of the conventional handpiece for an ultrasonic surgical instrument shown in FIGS. 12 and 1 are replaced with external cylinder 101 and horn 102, and a part of external cylinder 101 covering horn 102 except for these is quite similar to the conventional art shown in FIGS. 12 and 1. Then, in FIGS. 8A and 8B, only external cylinder 101 and horn 102 are shown, and description of the part of external cylinder 101 covering horn 102 except for these will be omitted.

FIGS. 8A and 8B are an outline view and a cross-section view of a tip portion of horn 102 protruding from external cylinder 101 and abutting against an affected area of the body.

Horn 102 of the present embodiment differs from conventional horn 1 having a constant external diameter, and is set to have the external diameter of the tip portion protruding from external cylinder 101 to be larger than the external diameter of the part covered by external cylinder 101 at the point of use. The external diameter of the tip portion of horn 102 is set to be slightly smaller than the inside diameter of external cylinder 101. This is because a procedure for assembling the handpiece for an ultrasonic surgical instrument is considered as suitable.

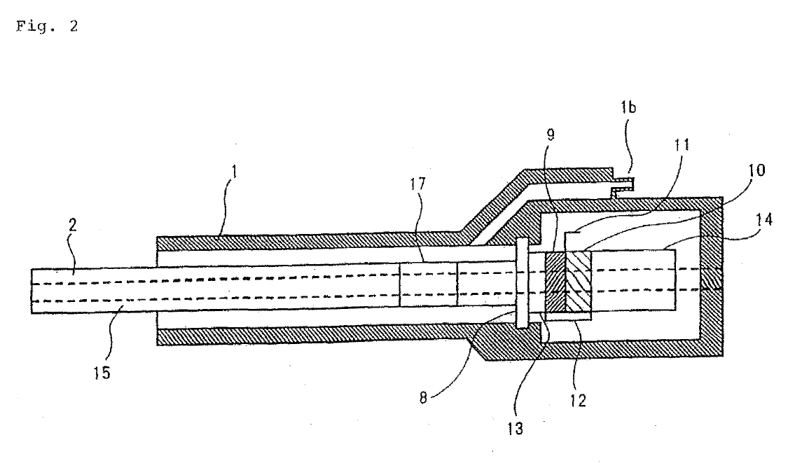

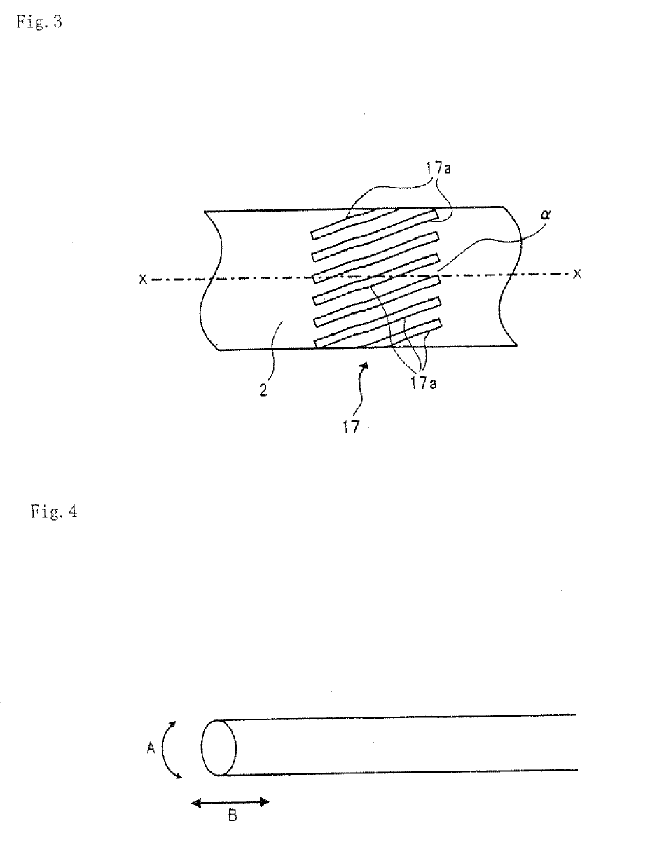

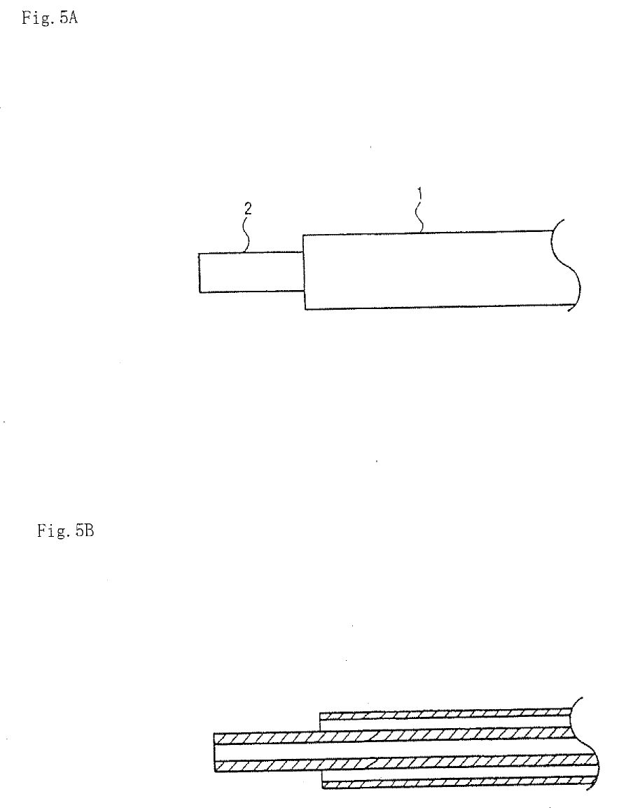

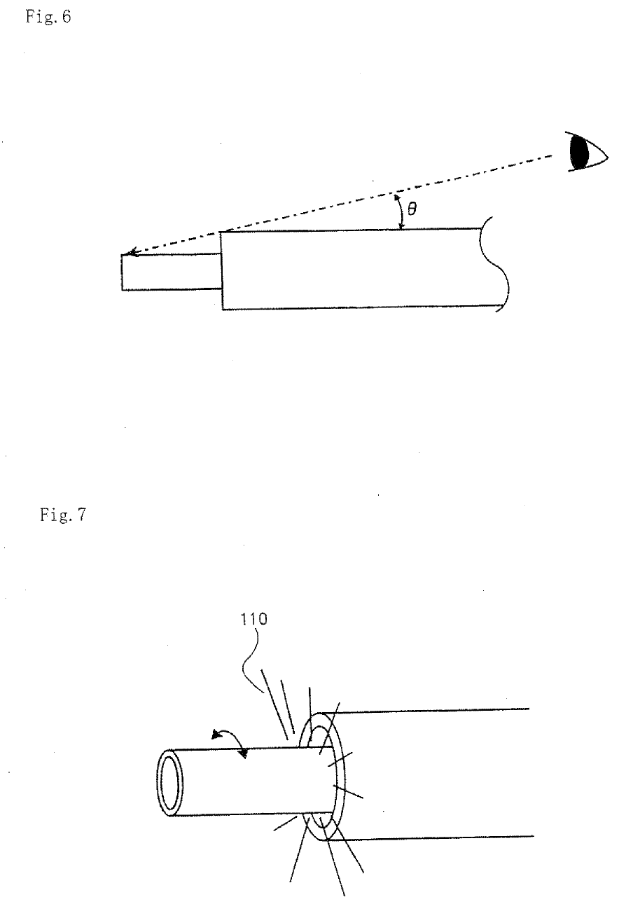

External cylinder 101 is divided into a part having the same external diameter and a part broadening in a conical shape (see FIG. 2), and these are screwed together so that they are unified. Horn 102 is screwed to flange 8 shown in FIG. 2. The external diameter of the tip portion of horn 102 is set to be slightly smaller than the inside diameter of external cylinder 101, and accordingly, after horn 102 is screwed into flange 8, external cylinder 101 can be assembled, and further, after external cylinder 101 is assembled, horn 102 can be also screwed into flange 8. In the present invention, because the relation between horn 102 and a part of external cylinder 101 covering horn 102 is important, the part covering horn 102 is hereinafter called “external cylinder 101”.

Because assembly can be implemented using any one of two sets of procedures for assembling as described above, workability can be improved at maintenance such as replacement of a part of components. In addition, when, after assembling external cylinder 101, horn 102 is to be screwed to flange 8, the external diameter of the tip portion of horn 102 can be set to be larger than the inside diameter of external cylinder 101, and further than the external diameter of external cylinder 101.

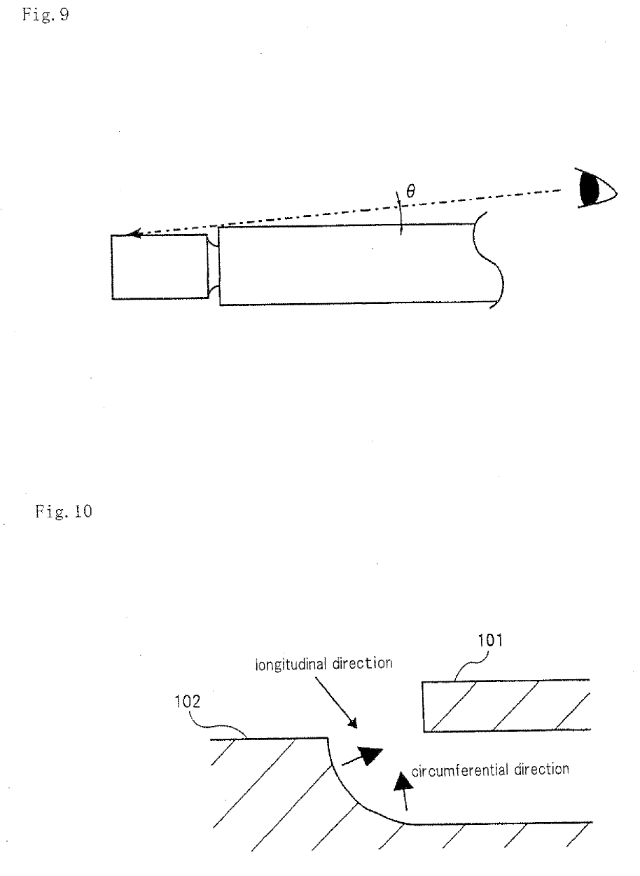

FIG. 9 illustrates an angle of visibility when the present embodiment is used for an operation.

As shown in FIG. 9, in the present embodiment, because the external diameter of the tip portion of horn 102 protruding from external cylinder 101 is larger than that of the conventional art, angle of visibility θ relative to the longitudinal direction of the handpiece turns out to be smaller, and therefore better visual recognition of the tip portion of horn 102 abutting against an affected area can be provided. This angle of visibility θ can be also made zero by configuring the external diameter of the tip portion of horn 102 to be larger than the inside diameter of external cylinder 101. Such configuration may be adopted.

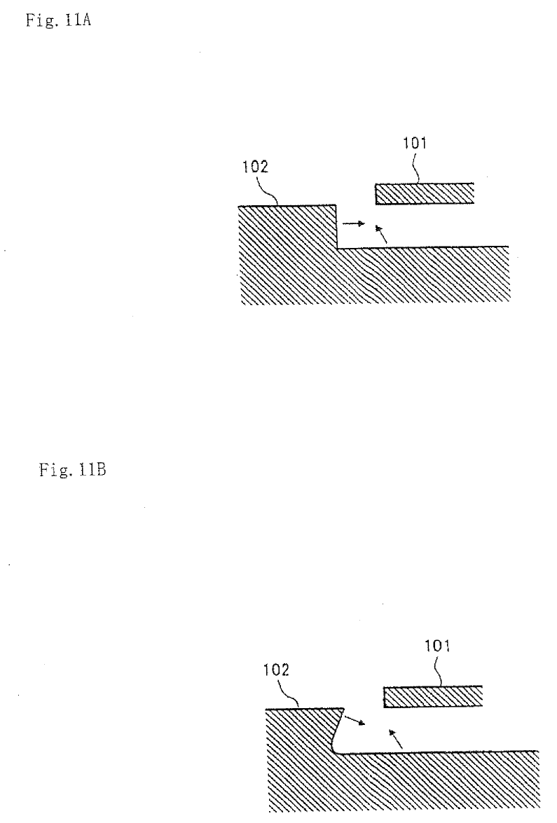

FIG. 10 illustrates a state of splash of irrigation water in the present embodiment, and is a cross-section view of an enlarged part surrounded by the dotted line in FIG. 8B.

In a joint portion between the tip portion of horn 102 and the part covered by external cylinder 101 that have different external diameter dimensions, a smooth curved surface is formed, and irrigation water moves from the right side to the left side as shown in FIG. 10. Because a component of the irrigation water that splashes off in the longitudinal direction (the horizontal direction as shown) due to reflection collides with the irrigation water that splashes off in the circumferential direction, the irrigation water flowing out from a gap between the external cylinder 101 and the horn 102 loses vigor thereof, as a result, the amount of splash in the circumferential direction is decreased, and does not act as an obstacle to view the tip portion of the horn.

FIGS. 11A and 11B are cross-section views illustrating a configuration of another embodiment of the present invention, and similarly to FIG. 10, illustrate a state of splash of irrigation water in each embodiment, and are cross-section views of the enlarged part surrounded by the dotted line in FIG. 8B.

In the embodiment shown in FIG. 11A, a joint portion between the tip portion of horn 102 and the part covered by external cylinder 101 that have different external diameter dimensions is formed to have a step. Irrigation water moves from the right side to the left side as shown in FIG. 11A. Because a component of the irrigation water that splashes off in the longitudinal direction (the horizontal direction as shown) due to reflection is made stronger, the irrigation water flowing out from a gap between the external cylinder 101 and the horn 102 more loses vigor thereof, and as a result, the amount of splash in the circumferential direction was decreased.

In the embodiment shown in FIG. 11B, a joint portion between the tip portion of horn 102 and the part covered by external cylinder 101 that have different external diameter dimensions is formed to have a tapered shape in which a tip portion is bored in a conical shape. Irrigation water moves from the right side to the left side as shown in FIG. 11B. Accordingly, the direction in which the irrigation water is reflected is made opposite to a gap portion between external cylinder 101 and horn 102 where the irrigation water flows out, and therefore a component of the irrigation water that splashes off in the longitudinal direction (the horizontal direction as shown) due to reflection is made further stronger than that shown in FIG. 11A, and then the irrigation water flowing out from a gap between the external cylinder 101 and the horn 102 further more loses vigor thereof, and as a result, the amount of splash in the circumferential direction is decreased.

FIG. 12 is a perspective view illustrating a configuration of another embodiment of the present invention. In the present embodiment, to further improve ablation of tissues with an end face of horn 102, grooves 501 are formed on the end face of horn 102 so that the end face is cut out in a cross shape. As configured as described above, better ablation of tissues was provided. Also, further better ablation of tissues was provided because irrigation water moves to the intake port through grooves 501.

It may be also thought that, to even further more improve ablation of tissues with an end face of horn 102, a flat surface of the end face is knurled to provide a nick. Such a configuration may be adopted.

Regarding the configurations of the embodiments described above, by possible combination of the configurations, for example, by combining formation of the step-like shapes shown in FIGS. 10 and 11 with formation of the groove shown in FIG. 12, the effects and advantages of each embodiment are synergistically enhanced. The present invention also includes a configuration provided by combining these configurations.

While the invention has been particularly shown and described with reference to exemplary embodiments thereof, the invention is not limited to these embodiments. It will be understood by those of ordinary skill in the art that various changes in form and details may be made therein without departing from the spirit and scope of the present invention as defined by the claims.

What is claimed is:

1. A handpiece for an ultrasonic surgical instrument, comprising:
a horn in which a flat surface is formed on an end face of a tip portion; and
an external cylinder for covering the horn except for the tip portion, wherein
an external diameter of the tip portion of the horn is larger than an external diameter of a part covered by the external cylinder.

2. The handpiece for an ultrasonic surgical instrument according to claim 1, wherein
the external diameter of the tip portion of the horn is larger than an external diameter of the external cylinder.

3. The handpiece for an ultrasonic surgical instrument according to claim 1, wherein
the horn is configured so that the tip portion and the part covered by the external cylinder are formed to have a smooth, curved surface.

4. The handpiece for an ultrasonic surgical instrument according to claim 1, wherein
the horn is configured so that a step is formed in a joint portion between the tip portion and the part covered by the external cylinder.

5. The handpiece for an ultrasonic surgical instrument according to claim 1, wherein
the horn is configured so that a joint portion between the tip portion and the part covered by the external cylinder has a tapered shape in which the tip portion is bored in a conical shape.

6. The handpiece for an ultrasonic surgical instrument according to claim 1, further comprising:
a groove formed on the end face of the tip portion.

7. The handpiece for an ultrasonic surgical instrument according to claim 1 further comprising:
a nick formed on the flat surface.

8. A horn, comprising a flat surface formed on an end face of a tip portion and constituting a handpiece for an ultrasonic surgical instrument along with an external cylinder for covering the horn except for the tip portion, wherein
an external diameter of the tip portion is larger than an external diameter of a part covered by the external cylinder.

9. The horn according to claim 8, wherein
the tip portion and the part covered by the external cylinder are formed to have a smooth, curved surface.

10. The horn according to claim 8, wherein
a step is formed in a joint portion between the tip portion and the part covered by the external cylinder.

11. The horn according to claim 8, wherein
a joint portion between the tip portion and the part covered by the external cylinder has a tapered shape in which the tip portion is bored in a conical shape.

12. The horn according to claim 8, further comprising:
a vibration conversion structure for converting ultrasonic longitudinal vibration into ultrasonic, torsional vibration.

13. The horn for an ultrasonic surgical instrument according to claim 8 further comprising:
a groove formed on the end face of the tip portion.

14. The horn for an ultrasonic surgical instrument according to claim 8, further comprising
a nick formed on the flat surface.
